(19) United States
(12) Patent Application Publication
(10) Pub. No.: US 2007/0155669 A1
(43) Pub. Date: Jul. 5, 2007

(54) Treatment of cancer by a combination of non-ionizing radiation and androgen deprivation

(75) Inventor: Ken Koshiba, Tokyo (JP)

Correspondence Address: KING & SPALDING LLP, 1180 PEACHTREE STREET, ATLANTA, GA, 30309-3521, US

(73) Assignee: Dornier MedTech Laser GmbH, Wessling (DE)

(21) Appl. No.: 11/395,038

(22) Filed: Mar. 31, 2006

(30) Foreign Application Priority Data
Dec. 30, 2005 (EP) EP05028738.2

(51) Int. Cl. A61K 38/09; A61K 31/138

(52) U.S. Cl. 514/15; 514/651

(57) ABSTRACT

A combined androgen deprivation and thermotherapy cancer treatment. For example, the combination can be used to treat prostate cancer. Chemical compounds, including a non-steroidal anti-androgen and/or an LH-RH agonist, such as androgen deprivation therapy (“ADT”), can be used in combination with application of non-ionizing radiation to treat cancer. The chemical compounds can be administered before, simultaneously with, and/or after the application of the non-ionizing radiation. When the ADT is applied before the application of the radiation, a reduction in cancer volume can be expected. In patients treated in such a manner, evidence of recurring cancer was not observed.

### RELATED PATENT APPLICATION

This patent application claims priority under 35 U.S.C. § 119 to European Patent Application No. EP05028738.2, entitled “Treatment of Cancer by a Combination of Non-Ionizing Radiation and Androgen Deprivation,” filed Dec. 30, 2005, the complete disclosure of which is hereby fully incorporated herein by reference.

### FIELD OF THE INVENTION

The invention relates generally to a new regimen of treating cancer, and more particularly to a new regimen of treating cancer with a combination of non-ionizing radiation and androgen deprivation.

### BACKGROUND OF THE INVENTION

At the beginning of the last century, cancer was the number seven cause of disease-based human death. By the end of the 1990s, it rose to number two. It is expected that cancer will soon be the number one cause of disease-based death. According to official estimations for Germany, 210,000 people died from cancer in 1997 and 338,000 people developed cancer in the same year.

Cancers differ significantly from each other. Some cancers grow fairly slowly; others grow rather rapidly. Some produce metastases; others do not. Some form solid tumors, whereas others, like leukemia, do not. The most frequently occurring forms of cancer are: carcinoma of the mamma, lung, stomach and prostate. Different types of cancer not only differ in their biology but also in their treatment regimen. Among the different types of cancer, carcinomas are the most targeted in cancer therapy.

There are three major approaches to cancer treatment: surgery, radiation and chemotherapy. Oftentimes, a combination of the different approaches may be advantageous. For example, tumor size may be first reduced by radiation and/or chemotherapy, and the tumor may subsequently be resected in surgery. Alternatively, tumor size may be first reduced by surgery and then further controlled or reduced by radiation and/or chemotherapy. In the chemotherapeutic approach, compounds that slow down or stop the growth of cells are frequently used. Such compounds are usually cytotoxic and destroy the cells. Radiation therapy and chemotherapy are marred by side effects as neither treatment is highly selective. It is challenging to obtain chemotherapeutic agents that selectively act on cancer cells but not on normal cells. It is also difficult to target radiation selectively to cancer cells but not to normal cells.

The above-described three major approaches are often supplemented by further measures such as the administration of hormones, the stimulation of the immune system, and the use of further adjuvants.

Although many treatment regimens are already known in the art, there is still a need for improving existing treatment regimens to reduce the side effects and/or to increase the efficiency of cancer destruction.

Observations of the therapeutic effect of heat on malignant tumors have been reported since the early ages. There are many reports in the early 1900s on the use of hyperthermia in treating cancer; and heat was thought to be the most important single factor in causing the regression of the tumors and even the cure of the patients (Rohdenburg G L. J. Cancer Res. 1917, 51:19-28). Hyperthermia in the early ages consisted mostly of incidental whole body hyperthermia caused by infectious diseases and high body temperature artificially induced with bacterial toxin. However, interest in the use of thermotherapy for treating cancer declined by the 1960s, possibly due to the disappearance of many infectious diseases with the introduction of antibiotics and technical difficulties associated with the practical application of hyperthermia.

As used herein, “thermotherapy” is synonymous with “thermoablation” and refers to the process of destroying tissues or cells using thermal energy.

The use of localized hyperthermia for treating prostate cancer using a microwave applicator was reported by Mendecki et al. in 1980 (Mendecki J, Friedenthal E, Botstein C. Int. J. Radiation Oncology Biol. Phys. 1980; 6:1583-8). Similar clinical trials have been reported by several groups by the late 1980s (Szmigielski S., Zielinski H., Stawarz B. et al. Urol. Res. 1998, 16:1-7; Servadio C, Leib Z., Prostate 1984, 5:205-11 Yerushalmi A, Servadio C, Leib Z et al., Prostate 1982; 3: 623-30, Roehrborn C G, Preminger G, Newhall P et al., Urology 1998; 51:19-28). However, the clinical efficacy was low and the treatment remained supplementary to radiotherapy and/or hormone therapy. The low efficacy was mainly due to the use of the transrectal route which only allowed an intraprostatic temperature of lower than 43° C. to be reached.

A new system, transurethral microwave thermotherapy (“TUMT”) (Devonec M, Tamura K, Perrin P. J. Endourol. 1993, 7:255-9), combines the heating of the prostatic tissue with the conductive cooling of the urethra, usually accomplished by arranging a chamber around the microwave antenna which is continuously perfused with cooled water. This urethral applicator reduces the temperature in the immediately adjacent tissue, allowing an even higher power to be used to create sufficient heat deep inside the prostatic tissue, while leaving the temperature of the urethral mucosa and the rectal wall within the safety range. It is generally accepted that microwave treatment can destroy prostatic tissue up to a radial distance of 16 mm, while maintaining innocuous urethral and rectal temperatures. Moreover, temperatures of 45° C. or higher for approximately 1 hour cause uniform thermoablation of the prostatic tissue (Larson T R, Bostwick D G, Corica A. Urology 1996, 47:463-9; Huidobro C, Balmsjo M, Larson T et al. J. Urol. 2004, 171:672-8).

Regarding the treatment of prostate cancer with TUMT, there are only a few reports up to the present (Khair A A, Pacelli A, Iczkowski K A et al. Urology 1999, 54:67-72; Larson B T, Bostwick D G, Corica A G, Larson T R. J. Urol. 2003, 170:12-9). They conclude that the effectiveness of TUMT for treating prostate cancer is disappointing mainly because of “the limitation of TUMT in reaching the peripheral prostate.” In the majority of their patients, TUMT was done shortly before radical prostatectomy without neoadjuvant hormone therapy (“NT”), and the mean volume of the prostate at the time of TUMT exceeded 53 ml which appeared too large to be treated by TUMT only. The radial distance from the urethral wall to the external margin of the fibrous capsule of the prostate with the volume of around 30 ml is usually within 15 mm, with an 18F microwave applicator placed in the urethra.

Adenocarcinoma is a major type of malignant tumors of the prostate; it is an androgen-dependent cancer. Androgen deprivation therapy (“ADT”) is specifically designed for this cancer and is more effective than any other chemotherapies.

Transitional cell carcinoma and sarcoma are other types of malignant tumors that originate from the prostate gland; they constitute less than 5% of malignant tumors of the prostate. ADT is not effective on these two types of malignant tumors. The term “prostate cancer” is used herein to refer to “adenocarcinoma of the prostate,” unless otherwise indicated.

Initially, bilateral orchiectomy (castration) was done as a hormone therapy to treat prostate cancer; later, estrogen (female hormone) was used. However, estrogen is notorious for its complications to cause cardiovascular insufficiency and liver dysfunction.

Use of LH-RH (luteinizing hormone-releasing hormone) agonist has the same effect as bilateral orchiectomy. LH-RH is produced by the hypothalamus and stimulates the pituitary gland to secrete LH, which stimulates the production of testosterone in the testes. However, excess dosage of LH-RH agonist causes suppression of LH secretion, which leads to medical castration. Less than 5% of the androgens are produced by the adrenal glands. These androgens can be blocked by the use of non-steroidal anti-androgens before these androgens are converted to dihydrotestosteron (DHT, the active species) in the prostate. The non-steroidal anti-androgens have fewer side effects than the steroidal anti-androgens.

ADT is synonymous with maximum androgen blockade (MAB), complete androgen blockade (CAB), and total androgen blockade (TAB).

ADT has been used before and after radiation therapy and prostatectomy in the treatment of prostate cancer. However, its effectiveness has been limited. It has been shown that reduction of androgen to castration levels by ADT reduces the size of the prostate and the tumor; when used before and during radiation therapy, it improves local control (Pilepich M V, et al. Int. J. Radiat. Oncol. Biol. Phys. 1995, 32:175-80) and reduces complications (Zelefsky M J, et al. Urology 1997, 49:38-45). However, overall survival of treated patients did not improve significantly, except in a single trial (The European Organization for Research and Treatment of Cancer multicenter randomized trial, Bolla M, et al. New Eng. J. Med. 1997, 337:295-300).

ADT has also been used before radical prostatectomy, a procedure termed neoadjuvant androgen deprivation or neoadjuvant hormone therapy (NHT). NHT has been shown to be an effective but palliative form of therapy (Cher M L, et al. Brit. J. Urol. 1995, 75: 771-777; Aus G, Abrahamsson P A, et al. BJU International 2002, 90: 561-6).

Therefore, one object underlying the invention was to provide further means for treating cancer, in particular, solid tumor-forming carcinomas.

### SUMMARY OF THE INVENTION

The invention relates generally to a new regimen of treating cancer, and more particularly to a new regimen of treating cancer with a combination of non-ionizing radiation and androgen deprivation.

In one exemplary embodiment, a non-steroidal anti-androgen and/or an LH-RH agonist, such as androgen deprivation therapy (“ADT”), is used in combination with application of non-ionizing radiation. The chemical compounds can be administered before, simultaneously with, and/or after the application of the non-ionizing radiation.

Such an embodiment provides a therapeutic combination for treating cancer, comprising: (a) a non-steroidal anti-androgen, and/or (b) LH, LH-RH, and/or an LH-RH agonist, and (c) non-ionizing radiation, wherein the anti-androgen and/or the agonist can be administered before, simultaneously with, and/or after the application of radiation to the patient. In the following, the use of the term “radiation” means “non-ionizing radiation,” unless expressly stated otherwise.

In one embodiment, the type of cancer to be treated is a carcinoma. For example, the cancer to be treated can be an adenocarcinoma, such as a prostate adenocarcinoma.

The term “non-steroidal anti-androgen” is used herein to refer to an agent which blocks the action of dihydrotestosterone (“DHT”), a compound that is structurally similar to testosterone and that stimulates protein synthesis in prostate cells and prostate cancer cells. In addition, such an agent would not have any steroidal effects. The non-steroidal anti-androgen is assumed to block the conversion of androgens produced by the adrenal glands to DHT in the prostate. For example, the non-steroidal anti-androgen can be bicalutamide and/or flutamide.

In another embodiment, the anti-androgen can be administered in a dosage in a range of between 10 and 1,000 mg per day. For example, the anti-androgen bicalutamide can be administered in a dosage in a range of 80-150 mg per day, and the anti-androgen flutamide can be administered in a dosage in a range of 250-375 mg per day.

The term “LH-RH agonist” is used herein to refer to a drug that is a synthetic analog of luteinizing hormone-releasing hormone (“LH-RH”), a hormone produced in the hypothalamus. LH-RH agonists mimic the function of LH-RH in triggering the pituitary to produce luteinizing hormone (LH), which in turn stimulates the production of testosterone. Because of the higher potency of the agonists, an excess of testosterone is initially produced. The body detects this excess of testosterone and responds by decreasing the production of LH-RH. Thus, after an initial increase in testosterone levels, the LH-RH agonist is assumed to down-regulate the production of testosterone in the testes in excess dosage. In exemplary embodiments, the LH-RH agonist can comprise leuprorelin acetate, goserelin acetate, buserelin acetate, or tripterelin.

In another embodiment, the agonist can be administered in a dosage amount in a range of 1-10 mg every 4 weeks. For example, agonist leuprorelin acetate can be administered in a dosage of 3.75 mg every 4 weeks, and agonist goserelin acetate can be administered in a dosage of 3.6 mg every 4 weeks. Alternatively, agonist leuprorelin can be administered in a dosage of 11.25 mg every three months, and agonist goserelin acetate can be administered in a dosage of 10.8 mg every three months.

In an exemplary embodiment, the patient can be treated with the non-steroidal anti-androgen and the LH-RH agonist (such as ADT) for at least about one month, or possibly for at least about three months, before the application of radiation is started.

In another embodiment, the anti-androgen and the agonist can be administered sequentially. For example, the anti-androgen can be administered first and the agonist can be administered second. In one embodiment, the anti-androgen can be administered first and the agonist can be administered about one to two weeks later. Alternatively, the non-steroidal anti-androgen can be administered first, and the LH-RH agonist can be administered second. In one embodiment, the agonist can be administered first and the anti-androgen can be administered about one to two weeks later. According to another embodiment, both compounds can be administered simultaneously.

The non-steroidal anti-androgen and the LH-RH agonist can be administered by the same or a different route. For example, the non-steroidal anti-androgen can be orally administered, and the LH-RH agonist can be subcutaneously administered.

In one embodiment, diethylstilbestrol (“DES”) can be administered when ADT becomes ineffective. For example, the DES can be administered at a dosage in a range of 100-500 mg per day, either orally or intravenously. DES is a synthetic analog of the female hormone estrogen. Administration of DES increases the level of sex hormones in the body. When the body detects the excess of sex hormones, it regulates the level of such hormones by stopping production of testosterone. This eventually leads to slower growth of prostate cancer cells.

In one embodiment, the application of radiation starts at least about four weeks after the start of treatment with the anti-androgen and/or the agonist. For example, there can be at least about three months between the start of the agonist treatment and the radiation.

In an alternative embodiment, the radiation is applied simultaneously with ongoing treatment of the anti-androgen and/or the agonist. For example, over the period of time during which the chemicals are administered, the radiation also can be applied. The chemicals and the radiation do not need to be administered/applied at the same time point of a day to be applied “simultaneously.”

In one embodiment treatment with the anti-androgen and the agonist continues after application of the radiation. For example, the treatment can continue for about one month to three months after application of the radiation.

In one embodiment, the radiation to be applied is heat-generating radiation. Thermal energy can be supplied to the tissue to be treated in the form of heat from the local absorption of applied microwave energy, radio frequency energy, or light energy, including energy from laser light. For example, microwave radiation to be administered can be in the range of about 800 to about 1500 MHz, about 800 to about 1300 MHz, or about 800 to about 1000 MHz.

In one embodiment, the radiation can be administered to the target tissue, such as the prostate, via a helical coil antenna. For example, the helical coil antenna can be enclosed in a transurethral delivery system, which can contain a water-cooling circuit system. Alternatively, the radiation can be applied by way of transurethral microwave thermoablation (“TUMT”).

In one embodiment, the energy to be administered to the target tissue can be within the range of about 25 to about 100 watts, about 25 to about 90 watts, or about 25 watts to about 85 watts.

In another embodiment, the radiation can be applied over a period of at least about one month and/or over a period of at least about three months. The radiation can be applied simultaneously with administration of the anti-androgen and/or the agonist. For example, over the period of time during which the chemicals are administered, the radiation also can be applied. The chemicals and the radiation do not need to be administered/applied at the same time point of a day to be applied “simultaneously.” For example, over the period of time during which the chemicals are administered, the radiation also is applied.

### DETAILED DESCRIPTION OF EXEMPLARY EMBODIMENTS

A new regimen of treating cancer includes a combination of non-ionizing radiation and androgen deprivation. In one exemplary embodiment, a non-steroidal anti-androgen and/or an LH-RH agonist, such as androgen deprivation therapy (“ADT”), is used in combination with application of non-ionizing radiation. The chemical compounds can be administered before, simultaneously with, and/or after the application of the non-ionizing radiation.

For example, the treatment can be used to treat prostate cancer. ADT employed before radiation is expected to reduce the total volume of the prostate in the majority of patients, rendering transurethral microwave thermoablation (“TUMT”) effective down to the peripheral prostate. Maximum reduction in prostate volume is expected in three months of ADT. Furthermore, three months of ADT may reduce the viability of the cancer cells, rendering TUMT more effective.

ADT employed after radiation is expected to accelerate apoptosis of remaining cancer cells which have been weakened by preceding ADT and TUMT. Transurethral resection of the prostate (TURP) in radical fashion three months after TUMT is expected to reveal the effectiveness of the ADT-radiation combination therapy.

A number of minimally invasive treatments have been developed in recent years for treating benign prostatic hyperplasia (BPH). Some of these new treatments employ the thermal effect of different energy sources on the prostatic tissue. The thermotherapies include TUMT, interstitial laser coagulation of the prostate (ILCP), high intensity focused ultrasound (“HIFU”), and transurethral needle ablation (TUNA). Among these treatments, HIFU and TUMT have been used to treat prostate cancer, whereas the other treatments have been thought to be unsuitable for treating cancer.

In HIFU, high intensity precision-focused ultrasound waves are used to heat and destroy targeted prostatic tissue. HIFU is for transrectal use; the tip of the probe is too large to be introduced into the urethra. HIFU can elevate tissue temperature in the focal zone up to 80-100° C. in a very short time (1-10 sec) while maintaining the interventional tissue temperature at a safe level. HIFU is considered a contact free “acoustic knife.” However, the focal zone of HIFU is restricted to a very small area at a time, and it thus takes a very long time (3-8 hours) to cover the entire prostate organ. Furthermore, general anesthesia is usually required. In addition, since the treatment temperature is very high, great care has to be taken when treatment is carried out near the external urethral sphincter or the peripheral prostate, just above the wall of the rectum, to avoid unnecessary tissue damage.

In TUMT, the heat energy is produced by a microwave generator with different microwave frequencies, depending on the machine used. Furthermore, different machines have different designs of the urethral applicator which have different heating profiles. A heating profile that covers substantially the whole prostate is necessary for effective TUMT. As a result, not all TUMT machines are suitable for carrying out the invention. In general, a microwave applicator having its antenna 5-10 mm away from the anchor balloon works well.

One type of TUMT machine, Urowave (made by Dornier Medizintechnik GmbH, which is now known as Dornier MedTech Systems GmbH), heats and kills cancer cells at 45° C. while leaving the adjacent organs outside the fibrous capsule of the prostate unharmed. The heat is delivered to the entire target tissue at one time, allowing the treatment time to be reduced to one hour as compared to HIFU. However, the effective depth of microwave using the Urowave machine is limited to about 15 mm from the surface of the urethral mucosa, rendering the device ineffective in treating tumors in an enlarged prostate. Therefore, it is advantageous to reduce the volume of the prostate by ADT before Urowave therapy. It is expected that about 90% of the patients with localized prostate cancer, who are candidates for radical prostatectomy or irradiation therapy, can be treated with this less invasive and more selective treatment modality.

There are two primary parameters in a microwave machine that can be adjusted for the purposes of prostate treatment-microwave frequency and power. Both change the total energy delivered to the prostate per unit of time. Adjusting power, which is defined as energy loaded per unit time, is a direct method of increasing (or decreasing) the amount of energy delivered. An indirect method of increasing (or decreasing) energy delivered is through the adjustment of frequency. Higher frequency is associated with more energy delivered, but at a lower penetration depth. In the specific case of the Urowave machine, microwave power of 25-90 Watts at frequencies of between 915-1296 MHz can be delivered.

To maintain a reasonable treatment time (usually an hour), more power is administered to larger prostates. However, the amount of power required to effect a treatment is not simply volume-dependent. A prostate with more fibromuscular tissue usually requires more power than one with more glandular tissue. The amount of power administered to each patient also depends on the temperature of the wall of the rectum adjacent to the prostate gland.

In one embodiment, laser thermotherapy is used instead of microwave thermotherapy. In laser thermotherapy, laser light of a specific wavelength is produced by a laser generator. The specific wavelength depends on the type of laser generator used. The laser generator is connected to a urethral applicator, which acts as an emitter of the laser light. The applicator is inserted transurethrally into the urethra and is kept in place via some physical device, such as an anchor balloon in the bladder. In one embodiment, the urethral applicator emits light in a diffused manner, such that the eventual spherical volume of light radiation encapsulates substantially the whole prostate gland. In another embodiment, the urethral applicator is incorporated in a transurethral delivery system, which can include a water-cooling circuit system to protect the integrity of non-target tissue.

For example, the emitted wavelength of the laser can be in a range of about 500 nm to 2200 nm, about 700 nm to 1600 nm, or about 800 nm to 1100 nm; the laser power to be applied can be in a range of about 10 W to 100 W or about 30 W to 60 W; and the duration of treatment can be in a range of 10 minutes and 60 minutes.

### EXAMPLES

The following examples further illustrate the exemplary embodiments:

### Example 1

### Treatment of Prostate Cancer by the Combination of Androgen Deprivation Therapy (ADT) and Transurethral Microwave Thermoablation (TUMT)

Patient Selection

A total of fourteen patients with biopsy-proven adenocarcinoma of the prostate, clinical stage T2 or less, during the period between September 2001 and February 2003, were enrolled. Bone scan did not identify metastasis to the bone in all patients. After thorough discussion with each patient, written informed consent was obtained. The mean age of the patients was 71.2 years (range 59-88). The mean serum prostate specific antigen (PSA) was 11.2 ng/ml (range 4.0-30.8). The mean volume of the prostate at initial transrectal ultrasound (TRUS) biopsy was 43.5 ml (range 21.8-85.0). Gleason scores were 6 or less in seven patients, 7 in six patients and 9 in one patient. Clinical stages were T1c in eleven patients and T2a in three patients. The patients and tumor characteristics are summarized below, in Table 1.

Androgen Deprivation Therapy (ADT)

The therapy started with oral administration of non-steroidal anti-androgen (bicalutamide 80 mg/day in thirteen patients and flutamide 375 mg/day in one patient) and followed with subcutaneous administration of LH-RH agonist (leuprorelin acetate 3.75 mg/4 wks) two weeks later. In four patients, however, both non-steroidal anti-androgen and LH-RH agonist were started simultaneously, with no evidence of flare-up. Transurethral microwave thermoablation (TUMT) was carried out at least twelve weeks (mean 14.6 weeks) after the initiation of LH-RH agonist so that satisfactory reduction in the volume of the prostate was achieved to render the thermoablation effective. The counting of length of ADT started at the first LH-RH agonist administration and terminated at the sixth month (i.e. 24 weeks) after TURP in radical fashion, the final operative procedure.

Transurethral Microwave Thermoablation (TUMT)

Urowave was used for TUMT. It is a second generation TUMT device, designed for the treatment of BPH (Trachtenberg J, Toi A, Yeung E, Habib F Application of Newer Froms of Therapeutic Energy in Urology. ISIS Medical Media, Oxford, 1995, 51-8; Roehrborn C G, Preminger C; Newhall P, et al. Urology, 1998, 51:19-28). It consists of a microwave power generator, a cooling system, and a control and monitoring system. The Urowave applies 915 MHz microwave energy to the prostate via a helical coil antenna enclosed in a transurethral delivery system, which also contains a water-cooling circuit system to provide 360° uninterrupted circumferential cooling to preserve urethral mucosa from heat injury. The diameter of the urethral applicator is approximately 18F when inflated with cooling water. Urethral applicator UA20 comprises a helical coil antenna of two cm in length and UA30 comprises a helical coil antenna of three cm in length. One can choose an appropriate one according to the length of the prostatic urethra of each patient. Besides, the urethral applicator is equipped with an anchor balloon of 10 ml capacity, approximately one cm proximal to the head of the helical antenna to keep the applicator in place during the treatment. In addition, the system features a rectal probe, with a series of three thermal couples mounted on the anterior midline (prostate aside) that continuously monitor the temperature at the rectal mucosa closest to the posterior aspect of the prostate. It serves as the key safety feature to prevent excessive heating of the rectal wall.

For the purpose of this study, the safety threshold was set at 44° C. in the urethral mucosa and at 42.5° C. in the rectum. With these settings, peak intraprostatic temperature reaches as high as 67° C. Mean temperature reaches a maximum of 55° C. at a radial distance of approximately 0.4 cm from the urethra and remained 45° C. or higher up to a distance of 1.5 cm. Fibrous capsule is a barrier to microwave, but it prevents excessive heating of the rectal wall and external urethral sphincter as well, preserving higher temperature to kill cancer cells inside the prostate. This system is capable of delivering up to 90 watts of power to the targeted tissue. The mean power used in this series was 48.6 watts (range 25-85).

The patients were subjected to a 60 minute treatment in an outpatient setting under local anesthesia with lidocaine jelly in the urethra, diclofenac sodium suppository (50 mg) in the rectum, and 10 ml of 2% lidocaine hydrochloride infused into the bladder cavity, before insertion of the urethral applicator for treatment. Indwelling catheter (14 F) was kept in place for 3 days. No patient complained of difficulty in urination after removal of the catheter.

Observed Reduction in Prostate Volume

Significant reductions in prostatic volume (mean 41.6%) were noted in all patients in three months with ADT, as listed below in Table 2. In another three months after TUMT and with continued ADT, reductions in prostate volume (mean 53.5%) were noted, as are shown in the Table 2. These reductions rendered TUMT satisfactorily effective to kill all the cancer cells in the majority of the patients and also made TURP in radical fashion easy and safe. The mean TURP weight of 14 patients was 13.4 grams.

Histopathological Features of the Therapy

With ADT for three months, TRUS biopsy specimens generally revealed progressive degenerative changes, but some fibromuscular hyperplasia with atrophic glands can be seen. In addition, some remnant of adenocarcinoma, though atrophied, but probably viable, can be detected. Three months after TUMT and continued ADT, TURP was carried out in radical fashion and all of the resected chips were subject to thorough histopathological study, which revealed remarkable degeneration with generalized fibrotic changes in the majority of the patients. No cancer cell was detected in 12 of 14 patients.

Two Patients who had Remnants of Cancer Cell in TURP chips

Case 10 is a 67 year-old who had a small firm nodule in the left lateral lobe of the prostate by digital rectal examination. Upon TRUS examination, volume of the total gland (TG) of the prostate measured approximately 21.8 ml, and transition zone (TZ) measured 7.7 ml, but no hypoechoic lesion was noted in the area where the nodule was felt. In the initial TRUS, 6 systematic biopsy revealed a well differentiated adenocarcinoma, Gleason score 2+2=4, in 2 biopsy cores from the left base and the middle area. Bone scintigraphy with ^{99m}Tc-phosphate did not reveal any metastasis to the bone. The decision for clinical staging was T2a. After ADT for fourteen weeks, the volume of the prostate reduced to 18.7 ml (14.2% reduction) and serum PSA level came down to 3.7 ng/ml (58.4% reduction). The repeated (second) TRUS biopsy at this time revealed atrophic change of the gland; but some well differentiated adenocarcinoma, Gleason score 2+2=4, were seen in biopsy core from the left base. Two weeks after the second TRUS biopsy, TUMT with Urowave using UA20 urethral applicator was carried out for 60 minutes, up to 50 watts. Twelve weeks after the TUMT and continued ADT, TURP in radical fashion was carried out; 7 grams of prostatic tissue were resected from the entire prostatic urethra, divided into 5 parts, median, anterior, left lateral, right lateral and apex. The TRUS measurement of prostatic volume immediately before the TURP was 12.2 ml (44% of original volume). Thorough histopathological study of all the resected chips revealed generalized and significant degenerative changes due to microwave heating throughout almost all the chips; however, a minor cancer lesion, probably viable, remained in a chip from the median bladder neck. The lesion was diagnosed as well differentiated adenocarcinoma, Gleason score 2+3=5. This area where the remaining lesion was detected is right beneath the anchor balloon of the urethral applicator of the Urowave and is a weak point of microwave thermoablation; it is also the easiest point to be thoroughly removed by TURP.

Case 12 is a 63 year-old who had a moderately enlarged, smooth-surfaced prostate. Serum PSA level was 6.3 ng/ml. Upon TRUS examination, the prostatic volume (TG) measured 33.6 ml and 6 systematic needle biopsy revealed moderately differentiated adenocarcinoma in 2 biopsy cores. One was a small cancer lesion from a core at the right middle zone, Gleason score of 2+1=3, and another one was in a core from the left apex, Gleason score of 3+4=7. Bone scintigraphy with ^{99m}Tc-phosphate did not reveal any metastasis to bone. The clinical stage was estimated as T1c. After ADT for 12 weeks, prostatic volume reduced to 18.5 ml (44.9% reduction) and serum PSA level came down to <0.2 ng/ml (96.8% reduction). TUMT with Urowave using UA20 urethral applicator was carried out for 60 minutes, up to 25 watts. Another 13 weeks after TUMT and with continued ADT, TURP in radical fashion was carried out and 5 grams of prostatic tissue were resected from the entire prostatic urethra, divided into 5 parts, median, anterior, left lateral, right lateral and apex. TRUS measurement of prostatic volume immediately before the TURP was 16.0 ml (52.4% reduction). Histopathological study revealed generalized and significant degenerative changes due to microwave heating, throughout almost all the chips. However, a minor cancer lesion, probably viable, was detected in a chip from the right lateral lobe of the prostate. The lesion was diagnosed as moderately differentiated adenocarcinoma, Gleason score 3+4=7. Though this lesion could have been removed by TURP in radical fashion, a second session of TUMT with Urowave using UA20 urethral applicator was carried out for 60 minutes, up to 35 watts, 8 weeks after the TURP. Serum PSA level remained at 0.1 ng/ml up to the present, 6 months after termination ADT. The patient has been doing well without any clinical evidence of tumor recurrence.

### Example 2

### Effect of the Neoadjuvant Hormone Therapy (NHT)

It is generally accepted that NHT reduces preoperative PSA nadir levels and positive margin rates significantly, but does not beneficially decrease the risk of PSA recurrence years after surgery. According to clinical experience, reduction in prostate volume was very significant (41.6% reduction) after three months of ADT. The resulting mean volume of 26.2 ml is small enough to be thoroughly heated by TUMT. In two patients (no. 9 and no. 14 in Table 2) whose prostate volume at initial examination were large, 85.0 and 73.6 ml, the volumes after three months of ADT still measured 47.0 and 45.5 ml, respectively. However, we could not find any cancer cell in their TURP chips, which weighed 31.0 and 24.0 grams, respectively. On the other hand, two patients, no. 10 and no. 12, who showed probably viable cancer cells in TURP chips, had smaller prostates, 12.2 and 16.0 ml, respectively, at TUMT. In patient no. 10, cancer cells were found at the base of the prostate close to the median bladder neck which is thought to be least sufficiently heated by TUMT because it is located just beneath the anchor balloon of the urethral applicator and which is the easiest location to be completely removed by TURP. In patient no. 12, probably viable cancer cells were found from TURP chips from the right lateral lobe. It is not entirely clear why these cancer cells survived the TUMT. However, the cancer cells were enclosed by a thick fibrous tissue layer. It is our expectation that all of the remnant cancer cells in these two patients were completely removed by the last surgical procedure, the TURP in radical fashion.

TABLE 1
Baseline characteristics of patients

| | | | Vol. | | | | |
|---|---|---|---|---|---|---|---|
| | | PSA | Prostate TRUS | | TRUS-biopsy | | Clinical |
| Case | Age | (ng/ml) | (ml) | Diff. | Gleason score | Location | stage |
| 1 | 69 | 30.8 | 35.1 | Mod | 4 + 3 = 7 | \textcircled{3}\textcircled{6} | T1c |
| 2 | 75 | 6.6 | 41.0 | Well | 2 + 3 = 5 | \textcircled{3} | T1c |
| 3 | 74 | 22.4 | 26.9 | Mod > Poor | 4 + 3 = 7 | \textcircled{1}\textcircled{2}\textcircled{3} | T1c |
| 4 | 88 | 5.2 | 47.0 | Well | 1 + 2 = 3 | \textcircled{1}\textcircled{3} | T1c |
| 5 | 66 | 8.7 | 35.0 | Well | 2 + 1 = 3 | \textcircled{1}\textcircled{6} | T1c |
| 6 | 76 | 10.9 | 31.9 | Mod | 3 + 4 = 7 | \textcircled{2} | T1c |
| 7 | 59 | 6.8 | 31.9 | Mod | 4 + 3 = 7 | \textcircled{4}\textcircled{5}\textcircled{6} | T2a |
| 8 | 76 | 4.0 | 35.9 | Mod | 3 + 2 = 5 | \textcircled{4}\textcircled{5}\textcircled{6} | T1c |
| 9 | 71 | 23.0 | 85.0 | Well > Mod | 3 + 4 = 7 | \textcircled{5} | T1c |
| 10 | 67 | 8.9 | 21.8 | Well | 2 + 2 = 4 | \textcircled{4} | T2a |
| 11 | 81 | 9.2 | 47.8 | Mod > Poor | 4 + 5 = 9 | \textcircled{5} | T2a |
| 12 | 63 | 6.3 | 33.6 | Mod | 2 + 1 = 3 | \textcircled{2} | T1c |
| | | | | | 3 + 4 = 7 | \textcircled{6} | |
| 13 | 75 | 5.0 | 43.6 | Well | 2 + 3 = 5 | \textcircled{5} | T1c |
| 14 | 69 | 9.4 | 73.6 | Mod | 3 + 3 = 6 | \textcircled{3}\textcircled{6} | T1c |
| Legend: “PSA” refers to prostate-specific antigen; “Well” refers to well differentiated; “Mod” refers to moderately differentiated; “Poor” refers to poorly differentiated; “TRUS” refers to transrectal | | | | | | | |
| # ultrasound; “\textcircled{1}” refers to right base; “\textcircled{2}” refers to right middle: “\textcircled{3}” refers to right apex; “\textcircled{4}” refers to left base; “\textcircled{5}” refers to left middle; and “\textcircled{6}” refers to left apex. | | | | | | | |

TABLE 2
Reductions in volume of the prostate measured with transrectal
ultrasound by androgen deprivation therapy (ADT) and transurethral
microwave thermoablation (TUMT)

| | | three | three | |
|---|---|---|---|---|
| | Before | months after | months after | TURP |
| | treatment | ADT | TUMT | wt |
| Case | TRUS (ml) | TRUS (ml) | TRUS (ml) | (g) |
| 1 | 35.1 | — | 14.6 | 5.0 |
| 2 | 41.0 | 24.6 | 21.6 | 16.0 |
| 3 | 26.9 | 21.3 | — | 2.5 |
| 4 | 47.0 | 34.0 | 29.3 | 18.0 |
| 5 | 35.0 | — | 20.2 | 10.1 |
| 6 | 31.9 | 19.4 | 20.0 | 15.0 |
| 7 | 31.9 | 15.4 | 7.4 | 2.8 |
| 8 | 35.9 | 19.6 | 14.6 | 10.2 |
| 9 | 85.0 | 47.0 | 41.7 | 31.0 |
| 10 | 21.8 | 18.7 | 12.2 | 7.0 |
| 11 | 47.8 | 22.5 | 13.6 | 8.0 |
| 12 | 33.6 | 18.5 | 16.0 | 5.0 |
| 13 | 43.6 | 22.5 | 15.7 | 10.0 |
| 14 | 73.6 | 45.5 | 37.0 | 24.0 |
| Mean | 44.8 | 26.2 | 20.8 | 13.4 |
| (range) | (21.8˜85.0) | (15.4-47.0) | (7.4-41.7) | (2.8-31.0) |

| | | |
|---|---|---|
| Reduction rate [symbol] | −41.6% | −53.5% |

Legend:
“TURP wt” refers to weight of the chips of transurethral resection of the prostate;
“ [symbol] ” indicates that cases 1, 3, and 5 were withdrawn due to insufficiency of data.

1. A method for treating cancer, comprising the steps of:
applying non-ionizing radiation to a cancer patient; and
administering at least one of a non-steroidal anti-androgen, a luteinizing hormone (“LH”), a luteinizing hormone-releasing hormone (“LH-RH”), and an LH-RH agonist to the patient.

2. The method according to claim 1, wherein the anti-androgen comprises one of bicalutamide and flutamide.

3. The method according to claim 1, wherein the administering step comprises administering the non-steroidal anti-androgen using a dosage in a range of 10 mg/day to 1000 mg/day.

4. The method according to claim 1, wherein the agonist comprises one of leuprorelin acetate, goserelin acetate, buserelin acetate, and tripterelin.

5. The method according to claim 1, wherein the administering step comprises administering the agonist using a dosage in a range of 1 mg/4 weeks to 10 mg/4 weeks.

6. The method according to claim 1, wherein the patient has prostate cancer, and wherein the applying step comprises applying the radiation to the patient's prostate gland.

7. The method according to claim 6, wherein the prostate cancer is an adenocarcinoma.

8. The method according to claim 1, wherein the administering step comprises orally administering the anti-androgen and subcutaneously administering the agonist.

9. The method according to claim 1, wherein the applying step occurs at least four weeks after the administering step.

10. The method according to claim 1, wherein the applying step occurs at least three months after the administering step.

11. The method according to claim 1, wherein the applying step occurs simultaneously with the administering step.

12. The method according to claim 1, wherein the applying step occurs before the administering step.

13. The method according to claim 1, wherein the applying step occurs after the administering step.

14. The method according to claim 1, wherein the administering step continues after the applying step for a period of at least one month.

15. The method according to claim 1, wherein the administering step continues after the applying step for a period of at least three months.

16. The method according to claim 1, wherein the non-ionizing radiation comprises heat-generating radiation.

17. The method according to claim 16, wherein the heat-generating radiation comprises one of microwave, laser light, and ultrasound.

18. The method according to claim 16, wherein the heat-generating radiation comprises microwave energy with a frequency range between 800 MHz to 1500 MHz.

19. The method according to claim 18, wherein the frequency range of the microwave energy is between 800 MHz and 1300 MHz.

20. The method according to claim 18, wherein the frequency range of the microwave energy is between 800 MHz and 1000 MHz.

21. The method according to claim 1, wherein the applying step comprises applying microwave radiation in a power range of 25 to 100 Watts to target tissue of the patient.

22. The method according to claim 21, wherein the power range of the microwave radiation is between 25 and 90 Watts.

23. The method according to claim 21, wherein the power range of the microwave radiation is between 25 and 85 Watts.

24. The method according to claim 1, wherein the applying step comprises applying microwave radiation by way of transurethral microwave thermoablation (TUMT).

25. The method according to claim 1, wherein the applying step comprises applying heat-generating microwave radiation for a period of between 10 minutes and 60 minutes.

26. The method according to claim 1, wherein the applying step comprises applying heat-generating radiation at a time of at least one month after a start of the administering step.

27. The method according to claim 1, wherein the applying step comprises applying heat-generating radiation at a time of at least three months after a start of the administering step.

28. The method according to claim 1, wherein the applying step comprises applying heat-generating radiation simultaneously with ongoing treatment of the administering step.

29. The method according to claim 1, wherein the applying step comprises applying heat-generating radiation in the form of laser radiation having a wavelength range of between 500 nm and 2200 mm.

30. The method according to claim 29, wherein the wavelength range of the laser radiation is between 700 nm and 1600 nm.

31. The method according to claim 29, wherein the wavelength range of the laser radiation is between 800 nm and 1100 nm.

32. The method according to claim 1, wherein the applying step comprises applying laser power in the range of 10 W to 100 W to target tissue of the patient.

33. The method according to claim 32, wherein the laser power range is between 30 W and 60 W.

34. The method according to claim 1, wherein the applying step comprises applying laser radiation for a period of between 10 minutes and 60 minutes.

35-39. (canceled)

40. A method for treating cancer, comprising the steps of:
applying non-ionizing radiation to a cancer patient; and
administering a non-steroidal anti-androgen to the patient in conjunction with the non-ionizing radiation.

41. The method according to claim 40, wherein the anti-androgen comprises one of bicalutamide and flutamide.

42. The method according to claim 40, wherein the administering step comprises administering the anti-androgen using a dosage in a range of 10 mg/day to 1000 mg/day.

43. The method according to claim 40 wherein the applying step is performed after the administering step.

44. The method according to claim 40, wherein the applying step is performed before the administering step.

45. The method according to claim 40, wherein the applying step is performed simultaneously with the administering step.

46. The method according to claim 40, wherein the patient has prostate cancer, and wherein the applying step comprises applying the radiation to the patient's prostate gland.

47. A method for treating cancer, comprising the steps of:
applying non-ionizing radiation to a cancer patient; and
administering an LH-RH agonist to the patient in conjunction with the non-ionizing radiation.

48. The method according to claim 47, wherein the agonist comprises one of leuprorelin acetate, goserelin acetate, buserelin acetate, and tripterelin.

49. The method according to claim 47, wherein the administering step comprises administering the agonist using a dosage in a range of 1 mg/4 weeks to 10 mg/4 weeks.

50. The method according to claim 47, wherein the applying step is performed after the administering step.

51. The method according to claim 47, wherein the applying step is performed before the administering step.

52. The method according to claim 47, wherein the applying step is performed simultaneously with the administering step.

53. The method according to claim 47, wherein the patient has prostate cancer, and wherein the applying step comprises applying the radiation to the patient's prostate gland.
